(19) United States
(12) Patent Application Publication
(10) Pub. No.: US 2024/0404648 A1
(43) Pub. Date: Dec. 5, 2024

(54) COUNTERFACTUAL GENERATION OF MOLECULAR CONFORMATIONS

(71) Applicants: Genentech, Inc., South San Francisco, CA (US); Hoffmann-La Roche Inc., Little Falls, NJ (US)

(72) Inventors: Natasa TAGASOVSKA, Basel (CH); Michael Robert MASER, South San Francisco, CA (US)

(21) Appl. No.: 18/680,089

(22) Filed: May 31, 2024

Related U.S. Application Data
(60) Provisional application No. 63/505,378, filed on May 31, 2023.
(60) Provisional application No. 63/508,596, filed on Jun. 16, 2023.

(51) Int. Cl. G16C 20/30; G16C 20/70

(52) U.S. Cl. CPC G16C 20/30; G16C 20/70

(57) ABSTRACT

A method may include determining a first conformer of a molecule. A first uncertainty metric of the first conformer of the molecule may be determined. A counterfactual generative model may be applied to generate a second conformer of the molecule associated with a second uncertainty metric. The counterfactual generative model may generate the second conformer by sampling from a latent space populated by a plurality of embeddings of molecular conformers. The molecular analysis model may be applied to determine, based on a structure of the second conformer, the molecular property of the molecule. Related systems and computer program products are also provided.

### CROSS REFERENCE TO RELATED APPLICATIONS

This application claims priority to U.S. Provisional Application No. 63/505,378, entitled “COUNTERFACTUAL GENERATION OF MOLECULAR CONFORMATIONS” and filed on May 31, 2023, and U.S. Provisional Application No. 63/508,596, entitled “COUNTERFACTUAL GENERATION OF MOLECULAR CONFORMATIONS” and filed on Jun. 16, 2023, the disclosures of which are incorporated herein by reference in their entireties.

### TECHNICAL FIELD

The subject matter described herein relates generally to molecular analysis and more specifically to machine learning enabled techniques for generating molecular conformations associated with low analytical uncertainty.

### INTRODUCTION

A molecule is a group of two more atoms held together by chemical bonds. Molecules form the smallest identifiable unit into which a pure substance can be divided while still retaining the composition and chemical properties of that substance. One example of a molecule is a small molecule having a molecular weight between approximately 100 Daltons and 1000 Daltons. Small molecule drugs, which modulate biochemical processes to diagnose, treat, and prevent a gamut of illnesses, have been a cornerstone in modern pharmacology due to a number of compelling advantages. For example, small molecule drugs are capable of penetrating cell membranes to reach intracellular targets. Moreover, small molecule drugs are adaptable to a wide variety of therapeutic applications. For instance, a small molecule drug may be formulated as pills and capsules, intravenous or subcutaneous injectables, inhalational medicines, or suppositories. The development of the small molecule drug may further extend to tailoring various pharmacokinetic properties including liberation, absorption, distribution, metabolism, and excretion.

### SUMMARY

Systems, methods, and articles of manufacture, including computer program products, are provided for generating molecular conformers for molecular machine learning (MolML) tasks. In one aspect, there is provided a system for determining the molecular property of a molecule that includes at least one processor and at least one memory. The at least one memory may include program code that provides operations when executed by the at least one processor. The operations may include: determining, for a molecule, a first conformer; determining a first uncertainty metric of the first conformer of the molecule; applying a counterfactual generative model to generate a second conformer of the molecule associated with a second uncertainty metric, where the counterfactual generative model generates the second conformer by at least sampling from a latent space populated by a plurality of embeddings of molecular conformers; and applying the molecular analysis model to determine, based at least on a structure of the second conformer, the molecular property of the molecule.

In another aspect, there is provided a method for determining the molecular property of a molecule. The method may include: determining, for a molecule, a first conformer; determining a first uncertainty metric of the first conformer of the molecule; applying a counterfactual generative model to generate a second conformer of the molecule associated with a second uncertainty metric, where the counterfactual generative model generates the second conformer by at least sampling from a latent space populated by a plurality of embeddings of molecular conformers; and applying the molecular analysis model to determine, based at least on a structure of the second conformer, the molecular property of the molecule.

In another aspect, there is provided a computer program product including a non-transitory computer readable medium storing instructions. The instructions may cause operations may executed by at least one data processor. The operations may include: determining, for a molecule, a first conformer; determining a first uncertainty metric of the first conformer of the molecule; applying a counterfactual generative model to generate a second conformer of the molecule associated with a second uncertainty metric, where the counterfactual generative model generates the second conformer by at least sampling from a latent space populated by a plurality of embeddings of molecular conformers; and applying the molecular analysis model to determine, based at least on a structure of the second conformer, the molecular property of the molecule.

In some variations, one or more features disclosed herein including the following features can optionally be included in any feasible combination.

In some variations, the counterfactual generative model may include an encoder trained to generate an embedding of the second conformer.

In some variations, the counterfactual generative model further includes a decoder trained to decode the embedding of the second conformer.

In some variations, the counterfactual generative model may be trained by at least training the encoder to generate the embedding of the second conformer such that the second conformer that is decoded from the embedding of the second conformer is similar to the first conformer but is associated with a different uncertainty metric than the first conformer.

In some variations, the counterfactual generative model may be trained over a plurality of training iterations until one or more convergence criteria are satisfied.

In some variations, the one or more convergence criteria may include reducing a loss function.

In some variations, the loss function may include a first term quantifying an uncertainty associated with a conformer generated by the counterfactual generative model.

In some variations, an uncertainty computation model trained to determine the uncertainty associated with the conformer generated by the counterfactual generative model may be applied.

In some variations, the uncertainty associated with the conformer may include an aleatoric uncertainty associated with data stochasticity

In some variations, the uncertainty computation model may determine the aleatoric uncertainty of the conformer by at least performing a simultaneous quantile regression (SQR) to determine an entire conditional distribution of different values of the molecular property determined by the molecular analysis model based on a structure of the conformer.

In some variations, the uncertainty computation model may determine the aleatoric uncertainty of the conformer by at least determining a standard deviation present in a posterior distribution of molecular property values inferred by the molecular analysis model performing a regression task to determine the molecular property of the molecule based on a structure of the conformer.

In some variations, the uncertainty associated with the conformer may include an epistemic uncertainty associated with the conformer being outside of a distribution of training data used to train the molecular analysis model.

In some variations, the uncertainty computation model may determine the epistemic uncertainty of the conformer by at least determining a metric quantifying a level of entropy across categorical distributions.

In some variations, the uncertainty computation model may include a plurality of linear classifiers. Each linear classifier may be trained to detect when the conformer is outside of a distribution of training data used to train the molecular analysis model. The uncertainty computation model may further include a machine learning model trained to determine the epistemic uncertainty of the conformer based at least on an output of each linear classifier.

In some variations, the uncertainty associated with the conformer may include an aleatoric uncertainty and an epistemic uncertainty.

In some variations, the loss function may further include a second term quantifying a similarity between the first conformer and the conformer generated by the counterfactual generative model.

In some variations, the second term may be a distance between the first conformer and the conformer generated by the generator model.

In some variations, the distance may be a root mean squared distance (RMSD).

In some variations, each training iteration of the counterfactual generative model may include applying the counterfactual generative model to generate a first counterfactual explanation having a first embedding of a third conformer of the molecule, evaluating, based at least on a loss function, the first counterfactual explanation, determining, based at least on the evaluating of the first counterfactual explanation, that one or more convergence criteria have not been satisfied, and in response to determining that the one or more convergence criteria have not been satisfied, updating the counterfactual generative model.

In some variations, each training iteration of the counterfactual generative model may further include in response to determining that the one or more convergence criteria have been satisfied, deploying the counterfactual model as a part of the counterfactual generative model.

In some variations, a subsequent training iteration of the counterfactual generative model may include applying the counterfactual generative model updated in a previous training iteration to generate a second counterfactual explanation having a second embedding of a fourth conformer of the molecule, and evaluating the second counterfactual explanation based at least on the loss function.

In some variations, the training of the counterfactual generative model may further include training a machine learning model to approximate the latent space.

In some variations, the machine learning model may include an encoder trained to generate an embedding for each molecular conformer included in training data. The machine learning model may further include a decoder trained to recover each molecular conformer by decoding the embedding.

In some variations, the training of the machine learning model may include applying the encoder to generate the embedding for each molecular conformer in the training data, applying the decoder to decode the embedding, and adjusting one or more parameters of the encoder and/or the decoder to reduce a distance between each molecular conformer in the training data and a corresponding decoding generated by the decoder.

In some variations, the machine learning model may be a variational autoencoder.

In some variations, the molecule may be a protein molecule, a small molecule, an ion, a nucleic acid, a polysaccharide, and/or a glycolipid.

In some variations, the molecular property may include one or more of a molecular structure, potency, clearance, permeability, expression, binding affinity towards a target molecule, non-specificity, stability, immunogenicity, non-toxicity, human-ness, and self-association.

In some variations, the molecular analysis model may be a machine learning model that has been trained to determine the molecular property of one or more molecules based at least on the structure of the one or more molecules.

In some variations, the molecular analysis model may be applied to determine the molecular property of the molecule based on the structure of the second conformer instead of a structure of the first conformer in order to reduce an uncertainty in an output of the molecular analysis model.

In some variations, one or more features comprising a structural difference between the first conformer and the second conformer may be identified. An uncertainty in an output of the molecular analysis model may be determined to be associated with the one or more features

In some variations, an uncertainty metric may quantify a level of uncertainty associated with the molecular property that is determined by the molecular analysis model for the molecule based on a corresponding conformer of the molecule.

In some variations, the second uncertainty metric may be different than the first uncertainty metric.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to reducing the uncertainty associated molecular machine learning (MolML) tasks, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a molecular analysis system, in accordance with some example embodiments;

FIG. 2 depicts a flowchart illustrating an example of a process for molecular analysis, in accordance with some example embodiments;

FIG. 3 depicts a flowchart illustrating another example of a process for generating a molecular conformer, in accordance with some example embodiments;

FIG. 4 depicts a schematic diagram illustrating an example of a process for generating an on-manifold molecular conformer with less predictive uncertainty, in accordance with some example embodiments; and

FIG. 5 depicts a block diagram illustrating an example of a computing system, in accordance with some example embodiments.

When practical, similar reference numbers denote similar structures, features, or elements.

### DETAILED DESCRIPTION

The molecular properties of a molecule may often be dependent on the three-dimensional structure of the molecule. For example, the binding affinity between a small molecule and a target molecule (e.g., a protein) depends on the ability of the small molecule to adopt a three-dimensional structure, or conformational shape, that is complementary to that of the target molecule. Similarly, the ability for antibodies to recognize and bind to certain antigens may also be dependent on the conformational states of the antibodies. For instance, under the early “lock-and-key” model of molecular recognition, binding occurs when an antibody interacts with an antigen exhibiting a rigid three-dimensional structure that is a perfect complementary match for the rigid three-dimensional structure of the antibody. According to the “induced-fit” paradigm that evolved therefrom, the presence of an antigen may induce conformational changes in the antibody such that the antibody adopts a three-dimensional structure that is complementary to that of the antigen. More recently, the concept of conformational selection posits that the conformation changes associated with an antibody interacting with an antigen originate from the conformational diversity that is present with a population of the antibody in the unbound state. That is, antibodies in the unbound state may explore different energy states (or energy levels) by assuming a variety of conformations, including higher-energy conformations that are structurally similar to the conformation of the antibody bound to the antigen. Through the course of binding, conformers that are structurally similar to the bound conformation of the antibody may be preferentially selected such that the overall antibody population shifts towards the bound conformation.

Modeling the conformational shapes of a molecule may be critical in many molecular machine learning (MolML) tasks, especially tasks in which one or more computation models (e.g., machine learning models and/or the like) are trained to learn the relationship between molecular properties and conformational shapes. Due to its flexible nature, a single molecule (e.g., a protein sequence, an arrangement of atoms, and/or the like) can exist as an ensemble of conformations in equilibrium with one another. Moreover, certain properties of the molecule may vary across the conformers in the corresponding conformational ensemble (CE). That is, a first conformer of the molecule may exhibit a first value of a property while a second conformer of the same molecule may exhibit a second, different value of the property. Examples of this property may include molecular structure (e.g., two-dimensional structure, three-dimensional structure, and/or the like), potency (e.g., biochemical potency, cellular potency, in vivo potency, and/or the like) clearance, permeability, expression, binding affinity towards a target molecule, non-specificity, stability, immunogenicity, non-toxicity, human-ness, self-association, and/or the like.

As used herein, the terms “conformer” and “molecular conformer” may be used interchangeably to refer a molecule with a same chemical composition as another molecule (or conformer) but differ in three-dimensional structure via rotations about one or more intramolecular bonds. That is, for the two molecules to be considered conformers (or molecular conformers) of one another, the difference in their respective three-dimensional structures may be reconciled to allow superimposition of the two molecules without breaking and reforming any intramolecular bonds. In the case of large molecules, such as protein molecules, the term “conformation” may refer to the spatial arrangement of the constituent atoms that determines the overall shape of the molecule. Since the chemical composition of a molecule may be represented by a two-dimensional connectivity graph, the conformer ensemble (CE) of the molecule may include three-dimensional structures that derive from the same two-dimensional connectivity graph. Moreover, in some cases, the three-dimensional structures in the conformer ensemble (CE) of the molecule may exhibit a pairwise root-mean-squared deviation (RMSD) satisfying one or more thresholds (e.g., RMSD≥0.1 Å). In some cases, the term “molecule” may refer to the corresponding conformer ensemble (CE), which contains the various conformers of the molecule. However, since different conformers of the same molecule may exhibit different molecular properties (or different values of a molecular property), the property of the molecule across the entire conformer ensemble (CE) may but is not necessarily the same as the property of any individual conformers of the molecule.

Structure-based computation models, which are trained to determine the molecular property of a molecule based on its structure, may be sensitive to variations in the three-dimensional structure of the molecule. That is, as noted, different conformers of the same molecule, which exhibit different three-dimensional structures, may also exhibit different molecular properties. This may be reflected in the output of a structure-based molecular analysis model that has been trained to determine the molecular property of a molecule based on the three-dimensional structure of its individual conformers. For example, the structure-based molecular analysis model may determine different molecular properties for different conformers of the same molecule. In some cases, in addition to different molecular properties, the output of the structure-based molecular analysis model for different conformers of the same molecule may be associated with different degrees of uncertainty. For instance, the output of the structure-based molecular analysis model may be associated with a higher degree of uncertainty when the molecular analysis model is applied to determine the molecular property of a molecule based on the three-dimensional structure of a conformer that is outside of the distribution of conformers encountered by the molecular analysis model during training. Although it may be desirable to reduce the uncertainty in the output of the structure-based molecular analysis model, doing so by exposing the molecular analysis model to a larger quantity of conformers during training is not a practicable solution due to the high cost associated with extensive, high-precision conformational generation. Absent any practical means to mitigate uncertainty, the output of conventional structure-based molecular analysis models may not be sufficiently reliable for high stakes applications such as drug discovery.

Various embodiments of the present disclosure reduce the uncertainty in the output of a structure-based molecular analysis model when the structure-based molecular analysis model operates on one conformer of a molecule by generating, as an alternative input for the structure-based molecular analysis model, a different conformer of the same molecule. In some example embodiments of the present disclosure, a counterfactual generative model may be trained to generate, for a first conformer of a molecule, a second conformer of the same molecule that is associated with a different uncertainty metric. For example, in some cases, the first conformer may be associated with a first uncertainty metric when a molecular analysis model is applied to determine a molecular property of the molecule based on a first structure of the first conformer. The counterfactual generative model may be applied to generate the second conformer, which is associated with a second uncertainty metric that is different (e.g., by being lower or higher) than the first uncertainty metric of the first conformer. That is, when the molecular analysis model is applied to determine the molecular property of the molecule based on a second structure of the second conformer, the output of the molecular analysis model may be associated with the second uncertainty metric. As described in more detail below, the counterfactual generative model may generate the second conformer by at least sampling from a latent space populated by a plurality of embeddings of molecular conformers. For example, in some cases, the embedding of a molecular conformer may correspond to a reduced dimension representation of the molecular conformer in which the three-dimensional structure of the molecular conformer is parameterized by latent variables (or features). Moreover, in some cases, the molecular analysis model may be applied to determine, based at least on a second structure of the second conformer, the molecular property of the molecule. Alternatively and/or additionally, one or more features forming the difference between the first three-dimensional structure of the first conformer and the second three-dimensional structure of the second conformer may be identified as contributing to the uncertainty present in the output of the molecular analysis model.

In some example embodiments, the counterfactual generative model may include a conformer generator model trained generate the second conformer to be similar to the first conformer but with a different (e.g., lower, higher, and/or the like) uncertainty metric than the first conformer. For example, in some cases, the training of the conformer generator model may include applying the conformer generator model to generate a plurality of counterfactual explanations until one or more convergence criteria are identified. In this context, each counterfactual explanation may correspond to a different molecular conformer of the molecule. Different molecular conformers may be considered counterfactual explanations by exhibiting structural differences that contribute to differences in other aspects of the molecular conformers, including the difference in the uncertainty level associated with each molecular conformer. One example of the convergence criteria include reducing (or minimizing) a loss function quantifying the uncertainty and the similarity (e.g., to the first conformer of the molecule) of the molecular conformer associated with each counterfactual explanation. For instance, in some cases, the conformer generator model may be applied to generate a first counterfactual explanation that is associated with a first loss before the conformer generator model is updated (e.g., the weights of the conformer generator model are updated) such that a second counterfactual explanation generated by the conformer generator model is associated with a second loss that is lower than the first loss. The conformer generator model may undergo multiple training iterations until the satisfaction of one or more convergence criteria such as the loss associated with the conformers generated by the conformer generator model converging to a minimum value. Once the conformer generator model is trained, the trained conformer generator model may be applied to generate the second conformer. Accordingly, while the second conformer generated by the trained conformer generator model remains structurally similar to the first conformer, the molecular analysis model may be determine the molecular property of the molecule with less uncertainty when the molecular analysis model is applied to determine the molecular property of the molecule based on the second structure of the second conformer than when the molecular analysis model is applied to determine the molecular property of the molecule based on the first structure of the first conformer.

In some example embodiments, an uncertainty computation model may be applied to determine the first uncertainty metric associated with the first conformer and the second uncertainty metric associated with the second conformer. For example, in some cases, the first uncertainty metric and the second uncertainty metric may include an epistemic uncertainty associated with the corresponding conformer being outside of a distribution of training data used to train the molecular analysis model. Accordingly, in some cases, the uncertainty computation model may include a collection of orthonormal certificates (OCs), each of which being a linear classifier that detects when a conformer is outside of the distribution of the training data, and a machine learning model (e.g., a neural network and/or the like) trained to determine whether the conformer is outside of the distribution of the training data based on the output of the orthonormal certificates. Alternatively and/or additionally, the first uncertainty metric and the second uncertainty metric may include an aleatoric uncertainty associated with the natural stochasticity of observations. In the context of the molecular analysis model, aleatoric uncertainty may arise from measurement discrepancies in which different values of the molecular property are observed for the same molecular conformer. Accordingly, in some cases, the uncertainty computation model may be a machine learning model trained to determine the aleatoric uncertainty of a particular conformer by performing simultaneous quantile regression (SQR) to determine the entire conditional distribution (e.g., all quantiles) of the molecular property for the conformer. In some cases, the first uncertainty metric and the second uncertainty metric may include the epistemic uncertainty as well as the aleatoric uncertainty associated with the corresponding molecular conformer.

In some example embodiments, the first uncertainty metric and the second uncertainty metric may be specific to the type of task being performed by the molecular analysis model, in order to determine the molecular property of the molecule based on the structure of the corresponding molecular conformer. For example, in cases where the molecular analysis model performs a classification task to determine the molecular property of the corresponding molecular conformer, the first uncertainty metric and the second uncertainty metric may correspond to a categorical cross-entropy indicative of the epistemic uncertainty that is associated with the corresponding conformer. Alternatively, in cases where the molecular analysis model performs a regression task in order to determine the molecular property of each molecular conformer, the first uncertainty metric and the second uncertainty metric may correspond to the posterior standard deviation (e.g., the standard deviation present in the posterior probability distribution of molecular property values) indicative of the aleatoric uncertainty that is associated with the corresponding molecular conformer.

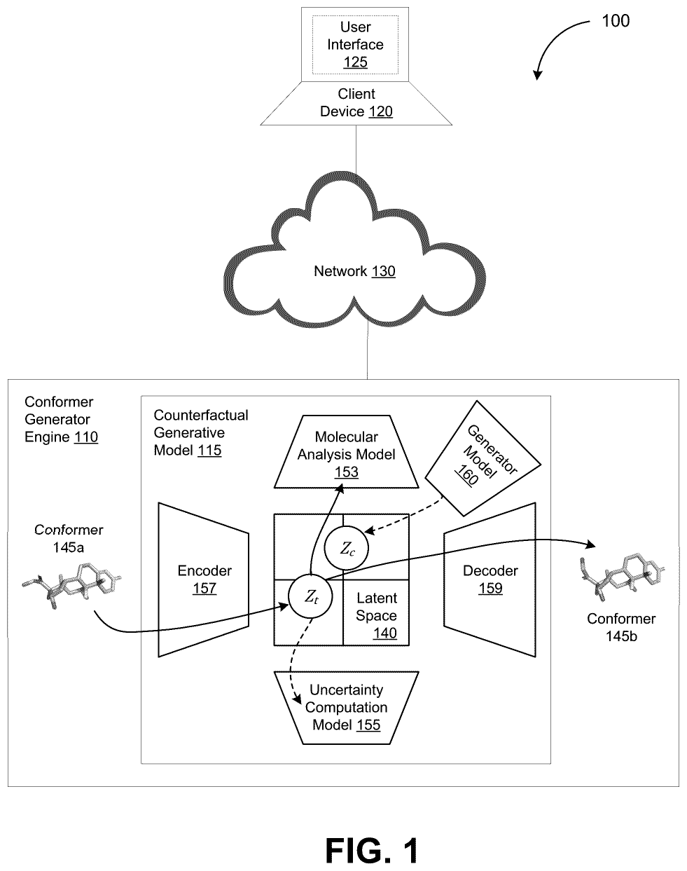

FIG. 1 depicts a system diagram illustrating an example of a molecular analysis system 100, in accordance with some example embodiments. Referring to FIG. 1 the molecular analysis system 110 may include a conformer generator engine 110 and a client device 120 communicatively coupled via a network 130. The client device 120 may be a processor-based device including, for example, a workstation, a desktop computer, a laptop computer, a smartphone, a tablet computer, a wearable apparatus, and/or the like. The network 130 may be a wired network and/or a wireless network including, for example, a local area network (LAN), a virtual local area network (VLAN), a wide area network (WAN), a public land mobile network (PLMN), the Internet, and/or the like.

Referring again to FIG. 1, the conformer generator engine 110 may include a counterfactual generative model 115 that samples a latent space 140 in order to generate, for a first conformer 145a having a first uncertainty metric, a second conformer 145b that is similar to the first conformer 145a but is associated with a second uncertainty metric that is lower than the first uncertainty metric of the first conformer 145a. For example, in some cases, the first conformer 145a may be associated with the first uncertainty metric when a molecular analysis model 153 is applied to determine the molecular property of a molecule based on a first structure (e.g., a first three-dimensional structure) of the first conformer 145a. As described in more details below, the molecular analysis model 153 (e.g., a multilayer perceptron (MLP) and/or the like) may be associated with various uncertainties (e.g., aleatoric uncertainty, epistemic uncertainty, and/or the like) that contributes to a lack of confidence in the value of the molecular property determined by the molecular analysis model 153. Examples of the molecular property include molecular structure (e.g., two-dimensional structure, three-dimensional structure, and/or the like), potency (e.g., biochemical potency, cellular potency, in vivo potency, and/or the like) clearance, permeability, expression, binding affinity towards a target molecule, non-specificity, stability, immunogenicity, non-toxicity, human-ness, self-association, and/or the like.

In this context, the term “molecule” may refer to a protein molecule or a non-protein molecule including, for example, a small molecule, an ion, a nucleic acid, a polysaccharide, a glycolipid, and/or the like. Examples or molecular properties determined by the molecular analysis model 153 may include binding affinity, absorption, distribution, metabolism, excretion, and/or the like. In some cases, the counterfactual generative model 115 includes a generator model 160, which may undergo multiple successive training iterations, each of which including the generator model 160 being updated to reduce (or minimize) a loss function quantifying the uncertainty and similarity (e.g., to the first conformer 145a) of a counterfactual explanation generated by the generator model 160.

In this context, the term “latent space” may refer to a data distribution that is populated with the embeddings of different molecular conformers. Each embedding may correspond to a reduced dimension representation of a corresponding molecular conformer in which the three-dimensional structure of the conformer is parameterized by latent variables (or features). It should be appreciated that the embedding of a molecular conformer (e.g., Zₜ or Z_{c} in FIG. 1) may parameterize the molecular conformer in fewer latent variables (or features) than the original feature space representation of the molecular conformer. Accordingly, the embedding of a molecular conformer may be considered a reduced dimension representation of the molecular conformer because the embedding has a fewer quantity of variables (or features) than the original feature space representation of the conformer. As shown in FIG. 1, in some cases, an encoder 157 (e.g., of an autoencoder such as a variational autoencoder (VAE)) may be trained to learn the latent variables for encoding the first conformer 145a such that the embedding of first conformer 145a (e.g., Zₜ in FIG. 1) can be decoded by a decoder 159 (e.g., of the autoencoder) to recover the original first conformer 145a. For example, the encoder 157 may map the first conformer 145a from its original representation in the feature space to an embedding (or reduced dimension representation) of the first conformer 145a (e.g., Zₜ in FIG. 1) in the latent space 140. Meanwhile, the decoder 159 decoding of the embedding (e.g., Zₜ in FIG. 1) may map the embedding of the first conformer 145a from the latent space 140 back to the original representation of the first conformer 145a in the feature space.

In some example embodiments, the generator model 160 may be trained to generate the second conformer 145b to be similar to the first conformer 145a but with a different (e.g., lower, higher, and/or the like) uncertainty metric than the first conformer 145a. That is, in some cases, the generator model 160 may be trained to generate the second conformer 145b to have a different uncertainty metric than the first conformer 145a but remain on the same data manifold (or data distribution) as the first conformer 145a. In some cases, the training of the generator model 160 may include multiple training iterations, each of which including the generator model 160 being applied to generate a counterfactual explanation, an evaluation of the counterfactual explanation based on a loss function (e.g., quantifying the uncertainty of the corresponding molecular conformer and similarity to the first conformer 145a), and an updating of the generator model 160 to reduce (or minimize) the loss function. For example, during each training iteration, the generator model 160 may be applied to generate a counterfactual explanation x_{c} while reducing (or minimizing) the loss function [symbol] shown as Equation (1) below. The generator model 160 may be trained upon satisfaction of one or more convergence criteria (e.g., the loss function [symbol] converging to a minimum). The trained generator model 160 may then be applied to generate the counterfactual explanation x_{CLUE}, which is associated with a minimum loss. This counterfactual explanation x_{CLUE} may be decoded (e.g., by the decoder 159) to generate the second conformer 145b in accordance, for example, based on Equation (2) below.

$$ℒ ( z ) = ( y \vert {μ}_{θ} ( x \vert z ) ) + d ( {μ}_{θ} ( x \vert z ) , {x}_{0} ) \qquad ( 1 )$$

$${x}_{CLUE} = {μ}_{θ} ( x \vert {z}_{CLUE} ) where {z}_{CLUE} = arg {min}_{z} ℒ ( z ) \qquad ( 2 )$$

Wherein the original observation, which is the first conformer 145a in this example, is denoted as x₀, [symbol] denotes some deferential estimate of uncertainty, and d(μ_{θ}(x|z), x₀) denotes the pairwise distance metric (e.g., root mean square distance (RMSD) and/or the like). In some cases, the pairwise distance metric d(μ_{θ}(x|z), x₀) can take the form of d (x, x₀)=λₓdₓ(x, x₀)+λ_{y}d_{y}(θ(x), θ(x₀)) in order to enforce similarity between the first conformer 145a and the second conformer 145b in both the feature space (e.g., of the first conformer 145a) and the aforementioned latent space. The hyperparameters (λₓ, λ_{y}) may control the tradeoff between a different (e.g., higher, lower, and/or the like) uncertainty metric and the similarity to the original input conformer x₀ (e.g., the first conformer 145a in the example shown in FIG. 1).

In some example embodiments, when implemented as a variational autoencoder (VAE), the encoder 157 may encode a variational latent space. This means that for each input, such as the first conformer 145a, the encoder 157 may output an embedding of the first conformer 145a by specifying the distribution of latent variables forming the embedding of the first conformer 145a. In some cases, the output of the encoder 157 a mean vector and a covariance matrix specifying the aforementioned distribution of latent variables. In some cases, the mean vector may specify the mean of each latent variable while the covariance matrix may indicate the covariance exhibited between each pair of latent variables. In some cases, the encoder 157 may generate the embedding of the first conformer 145a by updating the mean vector while the covariance matrix remains fixed (e.g., “fixed covariance”). However, doing so may decouple the gradients of the encoder 157 and the decoder 159, and diminish the ability of the decoder 159 to decode new molecular conformers from the embeddings generated by the encoder 157. Accordingly, in some cases, the encoder 157 may generate the embedding of the first conformer 145a by updating the mean vector as well as the covariance matrix such that the gradients of the encoder 157 and the decoder 159 remain coupled to improve the ability of the decoder 159 to decode new molecular conformers.

In some example embodiments, the molecular analysis model 153 may be applied to determine the molecular property of the molecule based on the structure of the second conformer 145b instead of the structure of the first conformer 145a in order to increase the certainty in the output of the molecular analysis model 153. The example in FIG. 1 shows that the molecular analysis model 153 may operate on the latent space representation of the second conformer 145b when determining the molecular property of the molecule. That is, the molecular analysis model 153 may operate directly on the embedding of the second conformer 145b output of the generator model 160 without any prior decoding of the embedding. However, it should be appreciated that the molecular analysis model 153 may also be trained to operate on the feature space representation of the second conformer 145b, in which case the decoder 159 may be first applied to decode the embedding of the second conformer 145b output by the generator model 160.

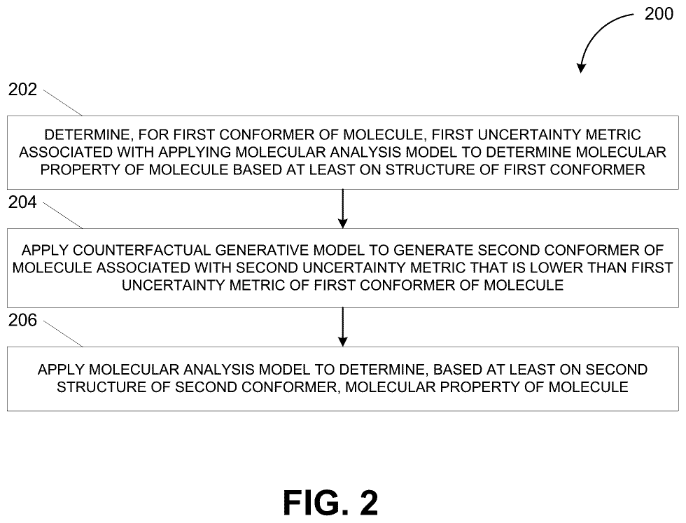

FIG. 2 depicts a flowchart illustrating an example of a process 200 for molecular analysis, in accordance with some example embodiments. Referring to FIGS. 1 and 2, the process 200 may be performed at the molecular generator engine 110, for example, to generate the second conformer 145b to be similar to the first conformer 145a but with a different uncertainty metric than the first conformer 145a.

At 202, a first uncertainty metric associated with applying a molecular analysis model to determine a molecular property of a molecule based at least on a first structure of a first conformer of the molecule is determined. In some example embodiments, a molecular analysis model may be applied to determine, based at least on a conformer of a molecule, a molecular property of the molecule. Examples of the molecular property include molecular structure (e.g., two-dimensional structure, three-dimensional structure, and/or the like), potency (e.g., biochemical potency, cellular potency, in vivo potency, and/or the like) clearance, permeability, expression, binding affinity towards a target molecule, non-specificity, stability, immunogenicity, non-toxicity, human-ness, self-association, and/or the like. However, the molecular analysis model may be associated with various uncertainties (e.g., aleatoric uncertainty, epistemic uncertainty, and/or the like) that contributes to a lack of confidence in the value of the molecular property determined by the molecular analysis model. As such, in some cases, a counterfactual generative model may apply an uncertainty computation model trained to determine the uncertainty metric associated with each individual conformer of the molecule. For example, in some cases, the uncertainty metric associated with a conformer of the molecule may correspond to a differentiable uncertainty quantification (UQ) associated with the molecular analysis model, which may include aleatoric uncertainty, epistemic uncertainty, and/or the like. This differentiable uncertainty quantification (UQ) may also be specific to the type of tasks being performed by the molecular analysis model in order to determine the molecular property of the molecule based on the structure (e.g., the three-dimensional structure) of the conformer. For instance, the uncertainty metric associated with the conformer of the molecule may be a categorical cross-entropy H(y|x), which quantifies the epistemic uncertainty that arise when the molecular analysis model performs a classification task in order to determine the molecular property of the molecule. Alternatively, the uncertainty metric associated with the conformer of the molecule may be a posterior standard deviation σ(y|x) (e.g., the standard deviation present in the posterior probability distribution of molecular property values), which quantifies the aleatoric uncertainty that arises when the molecular analysis model performs a regression task in order to determine the molecular property of the molecule.

In some cases, the uncertainty metric determined by the uncertainty computation model may account for an epistemic uncertainty associated with the conformer of the molecule being outside of a distribution of training data used to train the molecular analysis model. For example, in some cases, the uncertainty computation model may include a collection of linear classifiers (e.g., orthonormal certificates (OCs) and/or the like), each of which being capable of detecting when a conformer of the molecule is outside of the distribution of the training data. The uncertainty computation model may also include a machine learning model, such as neural network, that determines whether a conformer of the molecule is outside of the distribution of the training data based on the output of each linear classifier. For instance, in some cases, the machine learning model may determine, based at least on a variance in the outputs of the linear classifiers, when a conformer of the molecule is outside of the distribution of the training data used to train the molecular analysis model. Alternatively and/or additionally, the uncertainty metric determined by the uncertainty computation model may account for an aleatoric uncertainty associated with the natural stochasticity of observations (e.g., measurement discrepancies and/or the like). Accordingly, in some cases, the uncertainty computation model may be a machine learning model trained to determine the aleatoric uncertainty of the molecular analysis model by performing simultaneous quantile regression (SQR) to determine the entire conditional distribution (e.g., all quantiles) of molecular property values for a conformer of the molecule. In some cases, the degree of uncertainty associated with a conformer of the molecule may correspond to the spread of molecular property values associated with that conformer.

At 204, a counterfactual generative model is applied to generate a second conformer of the molecule associated with a second uncertainty metric that is lower than the first uncertainty metric of the first conformer of the molecule. In some example embodiments, a counterfactual generative model, which may include a trained generator model, may be applied to generate a counterfactual explanation corresponding to a different conformer of the molecule. In some cases, upon being trained to satisfy one or more convergence criteria, the generator model may be applied to generate another conformer of the molecule that exhibits less uncertainty when the structure of the conformer is used by the molecular analysis model to determine the molecular property of the molecule. In some cases, this conformer may be generated by at least decoding a corresponding embedding generated by the generator model.

At 206, the molecular analysis model is applied to determine, based at least on a second structure of the second conformer, the molecular property of the molecule. In some example embodiments, the molecular analysis model may be applied to determine the molecular property of the molecule based on the structure of the other conformer that is generated in operation 204. As noted, examples of the molecular property include molecular structure (e.g., two-dimensional structure, three-dimensional structure, and/or the like), potency (e.g., biochemical potency, cellular potency, in vivo potency, and/or the like) clearance, permeability, expression, binding affinity towards a target molecule, non-specificity, stability, immunogenicity, non-toxicity, human-ness, self-association, and/or the like. In some cases, the molecular analysis model may be a structure-based computation model exhibiting sensitivity to variations in the three-dimensional structure of the molecule. Accordingly, in some cases, the the molecular analysis model may be applied to determine the molecular property of the molecule based on the structure of a conformer of the molecule that is associated with less uncertainty. Furthermore, in some cases, the conformer generator engine may determine a difference between the structure of the different conformers of the molecule. The one or more features (e.g., structural features) forming the difference between the different conformers of the same molecule may be identified as contributing to the uncertainty in the output of the molecular analysis model.

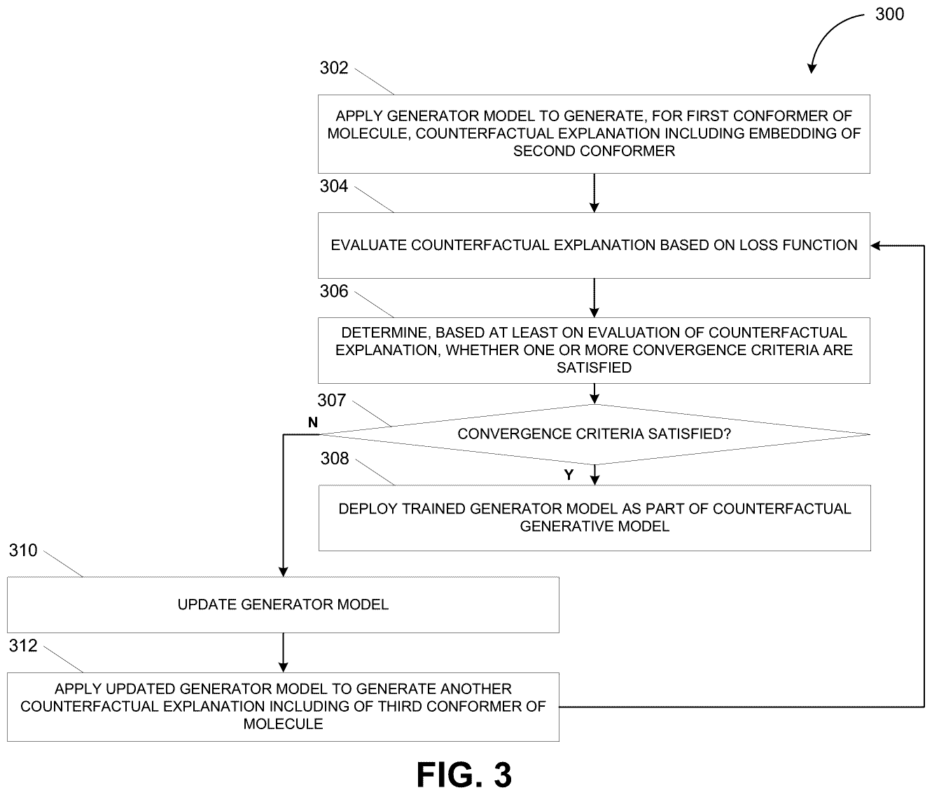

FIG. 3 depicts a flowchart illustrating an example of a process 300 for training the generator model 160, in accordance with some example embodiments. Referring to FIGS. 1-3, the process 300 may be performed at the conformer generator engine 110, for example, train the generator model 160 before the trained generator model 160 is deployed, as a part of the counterfactual generative model 115, to generate the second conformer 145b to be similar to the first conformer 145a but with a different uncertainty metric than the first conformer 145a.

At 302, a generator model is applied to generate, for a first conformer of a molecule, a counterfactual explanation including an embedding of a second conformer of the molecule. In some example embodiments, a generator model may undergo multiple successive training iterations, each of which being performed to update one or more parameters (e.g., weights, biases, and/or the like) of the generator model such that the generator model generates conformers that are similar but associated with less uncertainty. Accordingly, in some cases, each training iteration may include applying the generator model to generate a counterfactual explanation that includes an embedding of a molecular conformer. Moreover, as described in more details below, the generator model may be trained based on a loss function quantifying the uncertainty and the similarity of the molecular conformer associated with each counterfactual explanation until one or more convergence criteria, such as the reducing (or minimizing) of the loss function, are satisfied.

At 304, the counterfactual explanation is evaluated based at least on a loss function. In some example embodiments, upon generating a counterfactual explanation, the counterfactual explanation may be evaluated based on a loss function. In some cases, the loss function may include a first term quantifying the uncertainty associated with applying a molecular analysis model to determine the molecular property of the molecule based on the molecular conformer that is associated with the counterfactual explanation. Accordingly, in some cases, the counterfactual explanation may be evaluated by at least applying an uncertainty computation model to determine an uncertainty metric associated with applying the molecular analysis model to determine the molecular property of the molecule based on the structure of the corresponding molecular conformer. As noted, in some cases, the uncertainty metric may account for an aleatoric uncertainty and/or an epistemic uncertainty present in the output of the molecular analysis model. For aleatoric uncertainty, the uncertainty computation model may perform simultaneous quantile regression (SQR) to determine the entire conditional distribution (e.g., all quantiles) of different molecular property values determined by the molecular analysis model based on the structure of the molecular conformer. Meanwhile, for epistemic uncertainty, the uncertainty computation model may include a collection of linear classifiers (e.g., orthonormal certificates (OCs) and/or the like), each being capable of determining whether the molecular conformer is outside of the distribution of the training data used to train the molecular analysis model. The uncertainty computation model may further include a machine learning model (e.g., a neural network and/or the like) trained to determine, based on the output of each linear classifier, whether the molecular conformer is outside of the distribution of the training data used to train the molecular analysis model.

In some cases, the uncertainty metric determined by the uncertainty computation model may be specific to the type of tasks being performed by the molecular analysis model in order to determine the molecular property of the molecule based on the structure (e.g., the three-dimensional structure) of the molecular conformer. Accordingly, in some cases, the uncertainty metric determined by the uncertainty computation model may be a categorical cross-entropy H(y|x) metric, which quantifies epistemic uncertainty, where the molecular analysis model performs a classification task in order to determine the molecular property of the molecule. Alternatively, the uncertainty metric determined by the uncertainty computation model may be a posterior standard deviation σ(y|x) (e.g., the standard deviation present in the posterior probability distribution of molecular property values) metric, which quantifies aleatoric uncertainty, where the molecular analysis model performs a regression task in order to determine the molecular property of the molecule.

In some cases, the loss function may also include a second term quantifying the similarity between another conformer of the molecule with higher uncertainty and the molecular conformer associated with the counterfactual explanation. Accordingly, in some cases, the counterfactual explanation may also be evaluated by at least determining a similarity metric (e.g., a distance metric such as root mean square distance (RMSD) and/or the like) to quantify the similarity between the two molecular conformers. As described in more details below, the one or more convergence criteria may include reducing (or minimizing) a loss function quantifying the uncertainty and the similarity of the molecular conformer associated with the counterfactual explanation. Equation (1) is one example of this loss function in which the first term [symbol] corresponds to the uncertainty metric of a molecular conformer and the second term d corresponds to the similarity metric of the molecular conformer. Accordingly, in some cases, evaluating the molecular conformer x_{c} of each counterfactual explanation may include evaluating an informativeness of the conformer x_{c} based on the magnitude of uncertainty reduction, or the change in uncertainty Δ [symbol] _{gt}, relative to the original input conformer x₀ as indicated by Equation (3) below.

$$Δ {g t}_{} = {𝔼}_{{p}_{g t}} [ {g t}_{} ( y \vert {\overline{x}}_{0} ) - {g t}_{} ( y \vert {\overline{x}}_{c} ) ] \qquad ( 3 )$$

Furthermore, evaluating the molecular conformer x_{c} of each counterfactual explanation may include evaluating the relevance of the counterfactual explanation x, or the proximity of the conformer x_{c} the data manifold (or data distribution) of the original input conformer x₀. Accordingly, in some cases, the relevance of the conformer x_{c} may be quantified by a distance to the original input conformer x₀ (e.g., ∥Δx̅∥₁=∥x₀−x_{c}∥₁). Alternatively and/or additionally, where the generator model is a variational autoencoder, the relevance of the conformer x_{c} may also be quantified by the likelihood of the conformer x_{c} occurring in the output of the generative process performed by the generator model (e.g., log p_{gt}(x̅_{c})). It should be appreciated that reducing (or minimizing) the aforementioned loss function means that a conformer that is identified as satisfying the one or more convergence criteria may be associated with less uncertainty than another conformer of the same molecule when a molecular analysis model is applied to determine the molecular property of the molecule based on the structure (e.g., the three-dimensional structure) of that conformer. Moreover, by reducing (or minimizing) the aforementioned loss function, the resulting conformer may be relevant by retaining similarity (e.g., structural similarity) to other conformers of the molecule.

At 306, whether one or more convergence criteria are satisfied is determined based at least on the evaluation of the counterfactual explanation. As noted, in some example embodiments, multiple training iterations, each of which including applying the generator model to generate a counterfactual explanation, may be performed until one or more convergence criteria are satisfied. In some cases, the one or more convergence criteria may include reducing (or minimizing) a loss function having a first term corresponding to the uncertainty metric of a molecular conformer and a second term corresponding to the similarity metric of the molecular conformer. Accordingly, in some cases, the molecular conformer of each counterfactual explanation may be evaluated based on the corresponding uncertainty metric and the similarity metric. Reducing (or minimizing) the first term of the aforementioned loss function may maximize the informativeness of the conformer that is ultimately generated by the counterfactual generative model in terms of the magnitude of uncertainty reduction, or the change in uncertainty Δ [symbol] _{gt}, afforded by the conformer relative to other conformers of the molecule. Meanwhile, reducing (or minimizing) the second term of the loss function may maximize the relevance of the conformer, meaning that the structure of the conformer is relevant, for example, by being on the same manifold (or data distribution) as the other conformers of the molecule.

At 307—Yes, the one or more convergence criteria are satisfied. For example, in some cases, the one or more convergence criteria may be met when the loss function converges to a minimum value. Accordingly, at 308, upon determining that the one or more convergence criteria are met, the trained generator model is deployed as a part of a counterfactual generative model. For instance, once deployed, the counterfactual generative model may be applied to generate one or more conformers of a molecule. As noted, to generate a conformer of the molecule, the generator model may be applied to generate a corresponding embedding (e.g., a latent space representation of the conformer) before a decoder is applied to decode the embedding. In some cases, a molecular analysis model may be applied to determine the molecular property of the molecule based on the structure of the conformer instead of the structure of another conformer of the molecule at least because doing so increases the certainty in the output of the molecular analysis model. Alternatively and/or additionally, the difference (e.g., structural difference and/or the like) between the two conformers may be identified as contributing to the uncertainty in the molecular analysis model.

At 309—No, the one or more convergence criteria are not satisfied. For example, in some cases, the one or more converge criteria may not be satisfied if the loss function fails to converge to a minimum value. Accordingly, at 310, the generator model is further updated. For instance, in some cases, the generator model may undergo further updates that includes additional adjustments to one or more parameters (e.g., weights, biases, and/or the like) of the generator model. Furthermore, at 312, the updated generator model is applied to generate another counterfactual explanation that includes an embedding of a third conformer of the molecule. Thereafter, the process 300 may resume at operation 304 where the counterfactual explanation generated by the updated generator model is evaluated based on the loss function to determine whether one or more convergence criteria have been satisfied.

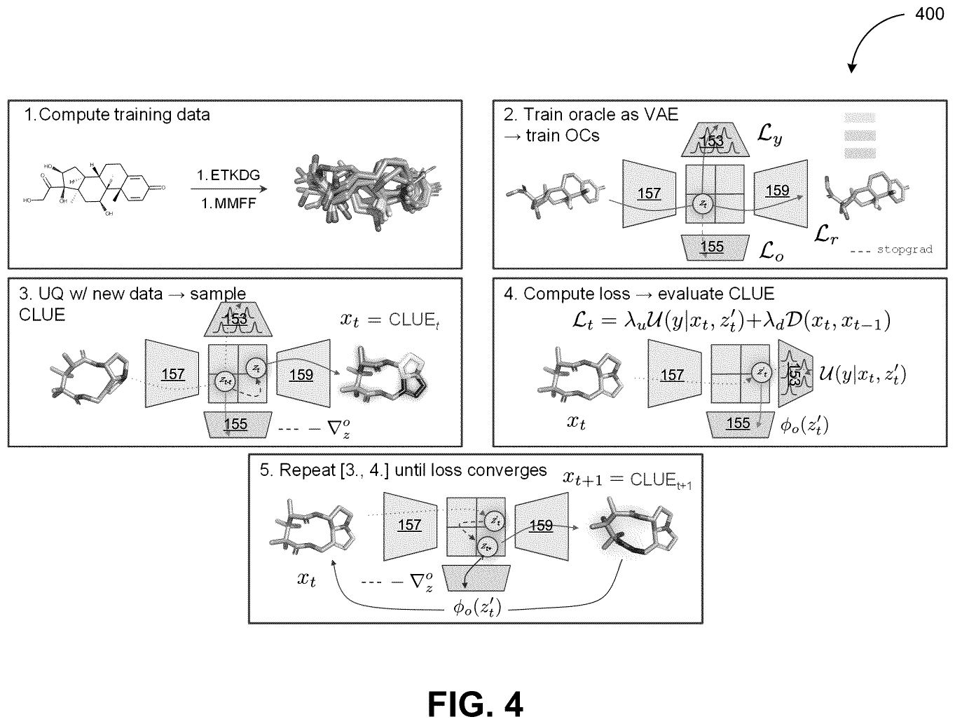

FIG. 4 depicts a schematic diagram illustrating an example of a process 400 for generating an on-manifold molecular conformer with less predictive uncertainty, in accordance with some example embodiments. Referring to FIG. 4, in Block 1 of the process 400, training data for a molecule may be generated. In the example shown in FIG. 4, the training data may include at least a portion of the conformer ensemble (CE) of a molecule. For example, as shown in FIG. 4, the generating of the training data may include applying, to a two-dimensional connectivity graph of the molecule, a conformer generating algorithm such as Experimental-Torsion basic Knowledge Distance Geometry (ETKDG) followed by Merck Molecular Force Field (MMFF).

Referring now to Block 2 of FIG. 4, the training data generated in Block 1 may be used to train the counterfactual generative model 160, which includes training the molecular analysis model 153, the uncertainty computation model 155, as well as the encoder 157 and the decoder 159 using the training data. For example, in some cases, the encoder 157 may be trained to generate an embedding for each molecular conformer in the training data that the decoder 159 can then decode to recover the original molecular conformer. In the example shown in FIG. 4, the training of the encoder 157 and the decoder 159 may include adjusting the encoder 157 and the decoder 159 to reduce (or minimize) the loss [symbol] ᵣ. Furthermore, in some cases, the molecular analysis model 153 and the uncertainty computation model 155 may be trained using the embedding of each molecular conformer included in the training data (e.g., generated by the encoder 157). For instance, in some cases, the molecular analysis model 153 may be trained to determine the molecular property of the molecule based on the structure of each molecular conformer (or an embedding thereof) included in the training data. Meanwhile, the uncertainty computation model 155 may be trained, based on the molecular conformers present in the training data, to approximate the distribution of the conformers in the training data. In the example shown in FIG. 4, the training of the molecular analysis model 153 may include updating the molecular analysis model 153 to reduce (or minimize) the loss [symbol] _{y} while the training of the uncertainty computation model 155 may include updating the molecular analysis model 155 to reduce (or minimize) the loss [symbol] ₒ. Once trained, the molecular analysis model 153 may determine the molecular property of the molecule based on the structure of its individual conformers whereas the uncertainty computation model 155 may determine the uncertainty present in the output of molecular analysis model 153 for each conformer. As noted, in some cases, the uncertainty computation model 155 may determine the uncertainty of the value of the molecular property output by the molecular analysis model 153 based on whether the molecular conformer used to determine the molecular property is within the distribution of the training data.

At Blocks 3-5 of the process 400, the generator model 160 is trained, over successive training iterations, to generate a counterfactual explanation, which when decoded (e.g., by the decoder 159) becomes a molecular conformer (e.g., the second conformer 145b) that is similar to an initial input molecular conformer (e.g., the first conformer 145a) but with less predictive uncertainty when the structure of the molecular conformer is used by the molecular analysis model 153 to determine the molecular property of the molecule. As shown in Block 3, the generator model 160 may be applied to generate one or more counterfactual explanations during each training iteration. In some cases, the generator model 160 may generate each counterfactual explanation CLUE, by at least generating the embedding zₜ of a corresponding molecular conformer xₜ. Meanwhile, in Block 4, each counterfactual explanation CLUE, may be evaluated based on the loss function [symbol] ₜ=λᵤ [symbol] ( [symbol] |xₜ, z′ₜ)+λ_{d} [symbol] (xₜ, xₜ₋₁), wherein [symbol] ( [symbol] |xₜ, z′ₜ) denotes the uncertainty metric associated with the molecular analysis model 153 and [symbol] (xₜ, xₜ₋₁) denotes the similarity metric relative to the original molecular conformer. As shown in Block 5 of FIG. 4, the training of the generator model 160 may continue over successive training iterations until one or more convergence criteria, such as the convergence of the loss [symbol] , are satisfied.

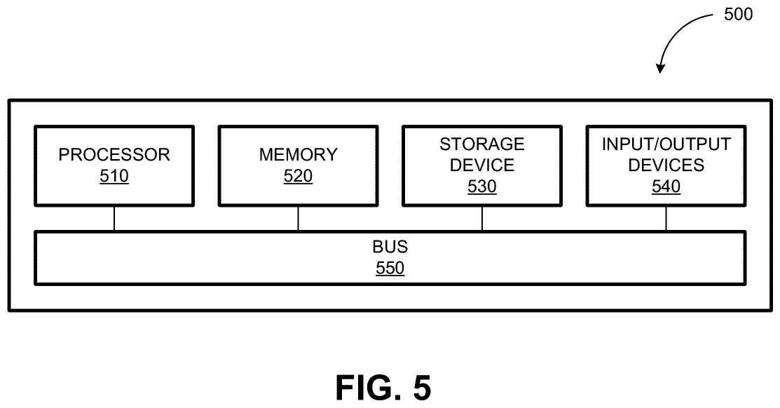

FIG. 5 depicts a block diagram illustrating an example of a computing system 500, in accordance with some example embodiments. Referring to FIGS. 1-5, the computing system 500 may be used to implement the conformer generator engine 110, the client device 120, and/or any components therein.

As shown in FIG. 5, the computing system 500 can include a processor 510, a memory 520, a storage device 530, and input/output devices 540. The processor 510, the memory 520, the storage device 530, and the input/output devices 540 can be interconnected via a system bus 550. The processor 510 is capable of processing instructions for execution within the computing system 500. Such executed instructions can implement one or more components of, for example, the conformer generator engine 110, the client device 120, and/or the like. In some example embodiments, the processor 510 can be a single-threaded processor. Alternately, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, or a tape device, or other suitable persistent storage means. The input/output device 540 provides input/output operations for the computing system 500. In some example embodiments, the input/output device 540 includes a keyboard and/or pointing device. In various implementations, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 500 can be used to execute various interactive computer software applications that can be used for organization, analysis and/or storage of data in various formats. Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions, which when executed by the at least one data processor, result in operations comprising:
determining, for a molecule, a first conformer;
determining a first uncertainty metric of the first conformer of the molecule;
applying a counterfactual generative model to generate a second conformer of the molecule associated with a second uncertainty metric,
where the counterfactual generative model generates the second conformer by at least sampling from a latent space populated by a plurality of embeddings of molecular conformers; and
applying a molecular analysis model to determine, based at least on a second structure of the second conformer, a molecular property of the molecule.

2. (canceled)

3. (canceled)

4. The system of claim 1, further comprising:
training the counterfactual generative model to generate the embedding of the second conformer such that the second conformer decoded from the embedding of the second conformer is similar to the first conformer but is associated with a different uncertainty metric than the first conformer, wherein the generator model is trained over a plurality of training iterations until one or more convergence criteria are satisfied.

5. (canceled)

6. The system of claim 4, wherein the one or more convergence criteria includes reducing a loss function that includes a first term quantifying an uncertainty associated with a conformer generated by the counterfactual generative model.

7. (canceled)

8. The system of claim 6, further comprising:
applying an uncertainty computation model trained to determine the uncertainty associated with the conformer generated by the counterfactual generative model.

9. The system of claim 8, wherein the uncertainty associated with the conformer includes an aleatoric uncertainty associated with data stochasticity.

10. The system of claim 9, wherein the uncertainty computation model determines the aleatoric uncertainty of the conformer by at least performing a simultaneous quantile regression (SQR) to determine an entire conditional distribution of different values of the molecular property determined by the molecular analysis model based on a structure of the conformer.

11. The system of claim 9, wherein the uncertainty computation model determines the aleatoric uncertainty of the conformer by at least determining a standard deviation present in a posterior distribution of molecular property values inferred by the molecular analysis model performing a regression task to determine the molecular property of the molecule based on a structure of the conformer.

12. The system of claim 8, wherein the uncertainty associated with the conformer includes an epistemic uncertainty associated with the conformer being outside of a distribution of training data used to train the molecular analysis model.

13. The system of claim 12, wherein the uncertainty computation model determines the epistemic uncertainty of the conformer by at least determining a metric quantifying a level of entropy across categorical distributions.

14. The system of claim 12, wherein the uncertainty computation model includes a plurality of linear classifiers, wherein each linear classifier is trained to detect when the conformer is outside of a distribution of training data used to train the molecular analysis model, and wherein the uncertainty computation model further includes a machine learning model trained to determine the epistemic uncertainty of the conformer based at least on an output of each linear classifier.

15. The system of claim 8, wherein the uncertainty associated with the conformer includes an aleatoric uncertainty and an epistemic uncertainty.

16. The system of claim 6, wherein the loss function further includes a second term quantifying a similarity between the first conformer and the conformer generated by the counterfactual generative model.

17. (canceled)

18. (canceled)

19. The system of claim 4, wherein each training iteration of the counterfactual generative model includes
applying the counterfactual generative model to generate a first counterfactual explanation having a first embedding of a third conformer of the molecule,
evaluating, based at least on a loss function, the first counterfactual explanation,
determining, based at least on the evaluating of the first counterfactual explanation, that one or more convergence criteria have not been satisfied, and
in response to determining that the one or more convergence criteria have not been satisfied, updating the counterfactual generative model.

20. (canceled)

21. (canceled)

22. (canceled)

23. The system of claim 4, wherein the counterfactual generative model comprises an autoencoder that includes an encoder and a decoder, wherein the training of the counterfactual generative model includes training the encoder to generate an embedding for each molecular conformer included in training data, and wherein the training of the counterfactual generative model further includes training the decoder to recover each molecular conformer by decoding the embedding.

24. The system of claim 23, wherein the training of the counterfactual generative model includes applying the encoder to generate the embedding of each molecular conformer in the training data, applying the decoder to decode the embedding, and adjusting one or more parameters of the encoder and/or the decoder to reduce a distance between each molecular conformer in the training data and a corresponding decoding generated by the decoder.

25. (canceled)

26. (canceled)

27. (canceled)

28. The system of claim 1, wherein the molecular analysis model comprises a machine learning model that has been trained to determine the molecular property of one or more molecules based at least on the structure of the one or more molecules.

29. The system of claim 1, wherein the molecular analysis model is applied to determine the molecular property of the molecule based on the structure of the second conformer instead of a structure of the first conformer in order to reduce an uncertainty in an output of the molecular analysis model.

30. The system of claim 1, further comprising:
identifying one or more features comprising a structural difference between the first conformer and the second conformer; and
determining that an uncertainty in an output of the molecular analysis model is associated with the one or more features.

31. The system of claim 1, wherein an uncertainty metric quantifies a level of uncertainty associated with the molecular property that is determined by the molecular analysis model for the molecule based on a corresponding conformer of the molecule.

32. The system of claim 1, wherein the second uncertainty metric is different than the first uncertainty metric.

33. A computer implemented system, comprising:
determining, for a molecule, a first conformer;
determining a first uncertainty metric of the first conformer of the molecule;
applying a counterfactual generative model to generate a second conformer of the molecule associated with a second uncertainty metric,
where the counterfactual generative model generates the second conformer by at least sampling from a latent space populated by a plurality of embeddings of molecular conformers; and
applying a molecular analysis model to determine, based at least on a second structure of the second conformer, a molecular property of the molecule.

34. A non-transitory computer readable medium storing instructions, which when executed by at least one data processor, result in operations comprising:
determining, for a molecule, a first conformer;
determining a first uncertainty metric of the first conformer of the molecule;
applying a counterfactual generative model to generate a second conformer of the molecule associated with a second uncertainty metric,
where the counterfactual generative model generates the second conformer by at least sampling from a latent space populated by a plurality of embeddings of molecular conformers; and
applying a molecular analysis model to determine, based at least on a second structure of the second conformer, a molecular property of the molecule.
